(19) United States
(12) Patent Application Publication
(10) Pub. No.: US 2005/0221417 A1
(43) Pub. Date: Oct. 6, 2005

(54) Sterility testing apparatus

(76) Inventors: Bronwen Houghton, Buckinghamshire (GB); Harold Capeling, Liverpool (GB)

Correspondence Address: OPPEDAHL AND LARSON LLP, P O BOX 5068, DILLON, CO, 80435-5068, US

(21) Appl. No.: 10/505,682

(22) PCT Filed: Feb. 21, 2003
(86) PCT No.: PCT/GB03/00750

§ 371 Date: May 10, 2005

(30) Foreign Application Priority Data
Feb. 28, 2002 (GB) 0204832.0
Nov. 1, 2002 (GB) 0225430.8

(51) Int. Cl. C12Q 1/04; C12M 1/34

(52) U.S. Cl. 435/34; 435/287.1

(57) ABSTRACT

A sterility testing apparatus for membrane filtration and direct inoculation sterility testing is disclosed. The apparatus has a test receptacle and preferably a sample receptacle. A sample flowpath directs the test sample to the test receptacle and a further flowpath directs wash and/or culture media to the test receptacle. The further flowpath has a microorganism filter arrangement upstream of the test receptacle to filter out microorganism contaminating the wash and/or culture media before it is introduced into the test receptacle. The possibility of “false positive” test results which indicate the presence of microorganisms when none, in fact, are present in the sample being screened, is therefore advantageously reduced compared to known sterility testing systems.

The present invention relates to screening samples for the presence of microorganisms.

In the pharmaceutical and food industries in particular, regulatory requirements often necessitate that products intended for human health administration or for human consumption are screened to ensure that the products are sterile or free from microbial (such as bacteria, fungi and moulds) contamination to assure that they are acceptable for their intended uses. This type of screening is often referred to as sterility testing.

Two different sterility testing methods, namely membrane filtration and direct inoculation, are used world-wide to test substances, preparations or articles which, according to the Pharmacopoeia, are required to be sterile. The preferred method of sterility testing is membrane filtration, however some samples cannot be filtered and must be tested using the direct inoculation method.

U.S. Pat. No. 4,036,698 discloses the industry standard membrane filtration sterility test, in which a sample (comprising the product to be screened, such as an antibiotic, optionally diluted in diluent or solvent) is allowed to flow through a cylinder having a membrane filter that filters out and traps microorganisms present in the sample. The cylinder (and membrane filter therein) is then flushed with a sterile solution. A microorganism growth culture medium, for example a soybean-casein digest medium, is introduced and retained in the cylinder such that the membrane filter is submerged in the growth culture medium. Visual observation of a colour change or turbidity of the growth culture medium after an appropriate incubation period at a suitable temperature indicates the presence of microorganisms in the test sample.

It is often required that a sample is screened for a number of different microorganisms that require different growth conditions. U.S. Pat. No. 4,036,698 discloses that aliquots of the sample can be introduced into more than one identical cylinder. Different microorganism growth culture media is introduced into each cylinder and the incubation time and incubation temperature of each cylinder can vary according to optimal growth conditions for the microorganisms of interest.

U.S. Pat. No. 5,213,967 discloses an automated membrane filtration sterility testing system in which a sample dissolution chamber, two growth media containers and a reservoir for providing diluent or solvent are all connected to two test canisters and two control canisters via a system of flexible tubes and pinch valves. The pinch valves direct flow of fluids (such as the sample and growth culture medium) to the test and control canisters at the appropriate time.

For direct inoculation sterility testing the sample to be tested is transferred directly into the appropriate growth culture medium. The inoculated culture medium is then incubated for an appropriate length of time at a suitable temperature to allow growth of microorganisms. Visual observation of a colour change or turbidity of the inoculated culture medium indicates the presence of microorganisms in the test sample.

One of the disadvantages of known sterility testing systems is the inherent possibility that contaminating microorganisms may be introduced into the system during the screening procedure even when the procedure is carried out in a clean room environment. Introduction of contaminating microorganisms may lead to “false positive” test results which indicate the presence of microorganisms when none, in fact, are present in the sample being screened.

To prevent contamination by foreign microorganisms, some sterility testing procedures incorporate use of formaldehyde or hydrogen peroxide gas to fumigate the external surfaces of the sterility testing apparatus. Alternatively or additionally, the growth culture medium canister(s) and test canister(s) may be placed in an isolator. Use of formaldehyde or hydrogen peroxide gas is potentially hazardous to the procedure operators, and use of an isolator is typically expensive.

An improved system for screening for microorganisms in a sample has now been devised.

According to the present invention, there is provided apparatus for screening for microorganisms in a sample, which apparatus comprises:
i) a test receptacle for growth/proliferation of microorganisms present in the sample;
ii) a sample flowpath to direct the sample to the test receptacle; and
iii) a further flowpath to direct wash and/or culture media to the test receptacle;
wherein, the further flowpath has a microorganism filter arrangement upstream of the test receptacle.

Preferably the further flowpath directs wash and culture media to the test receptacle. As the wash and/or culture media passes through the microorganism filter arrangement (typically a membrane filter no greater than 0.2 microns composed of cellulose acetate, cellulose nitrate or the like) upstream of the test receptacle, microorganisms (or at least microorganisms above a certain size) contaminating the wash and/or culture media are advantageously trapped by the filter arrangement before the wash and/or culture media is introduced into the test receptacle. The wash and/or culture media entering the test receptacle is therefore substantially sterile and free from microorganisms (or at least free from microorganisms above a certain size dictated by the pore size of the microorganism filter arrangement). The possibility of “false positive” test results which indicate the presence of microorganisms when none, in fact, are present in the sample being screened, is therefore advantageously reduced compared to known sterility testing systems.

The sample flowpath and the further flowpath may be separate flowpaths that are both respectively connected to the test receptacle. It is preferred, however, that the apparatus of the present invention includes a length of confluence flowpath comprising both the downstream portion of the sample flowpath and the downstream portion of the further flowpath, the confluence flowpath being connected to the test receptacle. In the latter preferred embodiment, the microorganism filter arrangement is provided in the further flowpath branch upstream of the confluence flowpath and not in line with the sample flowpath.

The sample flowpath, the further flowpath and the confluence flowpath may comprise one or more flexible tube(s). One or more valves (such as a pinch valve that acts externally on the flexible tube to releasably seal the tube) can be provided to control flow of fluids through the flexible tube(s) to the test receptacle. One or more pump(s) (such as a peristaltic pump) may be provided to induce flow of fluids through the flexible tube(s) to the test receptacle.

It is a preferred feature of the present invention that the sample flowpath includes one or more sample receptacles (typically of transparent plastic) arranged and configured to receive the sample or samples to be screened. The sample receptacle is in fluid communication with and upstream of the test receptacle. The sample can advantageously be stored and transported within the sample receptacle, until such time and place that screening of the sample is carried out.

The further flowpath preferably includes at least one media receptacle arranged and configured to receiving wash and/or culture media, the media receptacle being in fluid communication with and upstream of the test receptacle, wherein the microorganism filter arrangement is provided:
a) internally of the media receptacle; and/or
b) in the further flowpath downstream of the media receptacle but not in-line with the sample flowpath.

It is preferred that the microorganism filter arrangement is provided internally of the media receptacle.

The media receptacle is typically of transparent plastic with the microorganism filter arrangement being sealed at its periphery to the inner wall of the receptacle such that the filter arrangement spans the flowpath of fluid through the media receptacle.

The media receptacle is preferably arranged and configured to receive both wash and culture media. Provision of a common media receptacle for receiving both wash and culture media advantageously reduces costs and simplifies the sterility testing apparatus.

When the apparatus of the present invention includes a confluence flowpath substantially as hereinbefore described, the media receptacle is provided in the further flowpath branch upstream of the confluence flowpath and not in line with the sample flowpath.

According to a preferred embodiment of the present invention, the test receptacle has a sample microorganism filter arrangement therein for filtering microorganisms from the sample. This preferred embodiment is suitable for membrane filtration sterility testing.

Therefore, according to a second aspect of the present invention there is provided apparatus for screening for microorganisms in a sample, which apparatus comprises:
a) a test receptacle having a sample microorganism filter arrangement therein for filtering microorganisms from the sample;
b) a sample flowpath to direct the sample to the test receptacle; and
c) a further flowpath to direct wash and/or culture media to the test receptacle;
wherein, the further flowpath has a microorganism filter arrangement upstream of the test receptacle.

The sample microorganism filter arrangement is typically sealed at its periphery to the inner wall of the test receptacle such that the filter arrangement spans the flowpath of fluid through the test receptacle. The sample microorganism filter arrangement may be a membrane filter no greater than 0.45 microns composed of cellulose nitrate, cellulose acetate or the like. The membrane filter may be formed with an annular border of hydrophobic material on at least its upper surface and preferably on both surfaces, in order to prevent wetting of the membrane filter by the sample in the area of the seal.

Two or more test receptacles may be provided in the apparatus of the present invention. When two or more test receptacles are provided, the sample flowpath directs the sample to be screened to the two or more test receptacles, typically by use of a system of flexible tubes in which a first tube is connected to the sample receptacle at one end, the other end of the first tube being in fluid communication with two or more further tubes that are respectively connected to each test receptacle.

Furthermore, when two or more test receptacles are provided in the apparatus of the present invention substantially as hereinbefore described, the further flowpath typically directs wash and/or culture media to the two or more test receptacles, generally by use of a system of flexible tubes in which a first tube is connected to the media receptacle at one end, the other end of the first tube being in fluid communication with two or more further tubes that are respectively connected to each test receptacle.

Valves (such as pinch valves substantially as hereinbefore described) may be provided to each of the first tubes and further tubes of the sample and further flowpath to control flow of fluids to the test receptacles.

Vent ports may be provided in the test receptacle, sample receptacle and media receptacle. The vents port(s) preferably include a hydrophobic microporous filter typically of cellulose esters coated with a hydrophobic material. The filter is provided to filter all microorganisms above a specific size from the air flow through the filter.

The test receptacle, sample receptacle and media receptacle may be provided with volumetric calibration marks to indicate the volume of fluid in each receptacle. The different receptacles may also be provided with different labels, for example a number, name or colour code, to aid identification of the different receptacles and simplify the sterility testing procedure.

The apparatus of the present invention preferably includes a support member that releasably supports the test receptacle. The test receptacle can thus be released from the support member for incubation following introduction of culture medium into the test receptacle as hereinafter described in more detail. The support member may further support (typically releasably support) the sample receptacle and/or media receptacle when these are present in the apparatus of the present invention.

A control system may be used to automate flow of fluids to the test receptacle. The control system typically comprises a programmable control unit mounted on the support member, the control unit being connected to one or more actuators incorporated in the support member. The actuator(s) typically act on valves (such as pinch valves substantially as hereinbefore described) and/or pumps (such as peristaltic pumps substantially as hereinbefore described). The control unit can thus be programmed to spatially initiate the actuator(s) to control flow of fluids to the test receptacle.

The apparatus of the present invention is preferably an integral system which may be packaged within at least one (preferably two) substantially sealed bags for transportation and storage.

There is further provided by the present invention an integral sterility testing system comprising an apparatus for screening for microorganisms in a sample, the apparatus being substantially sealed within a bag.

It is a preferred feature of the present invention, that the apparatus of the integral sterility testing system is substantially sealed within an inner bag, the inner bag being substantially sealed within an outer bag. The outer bag is typically opened on aseptic transfer to a clean room environment and the inner bag may be opened at the sampling point.

The apparatus of the integral sterility testing system may comprise:
i) a test receptacle for growth/proliferation of microorganisms present in the sample being screened;
ii) a sample flowpath to direct the sample to the test receptacle; and
iii) a further flowpath to direct wash and/or culture media to the test receptacle; the further flowpath having a microorganism filter arrangement upstream of the test receptacle.

Alternatively, the apparatus of the integral sterility testing system may comprise:
i) a test receptacle having a sample microorganism filter arrangement therein, for filtering microorganisms from the sample being screened;
ii) a sample flowpath to direct the sample to the test receptacle; and
iii) a further flowpath to direct wash and/or culture media to the test receptacle; the further flowpath having a microorganism filter arrangement upstream of the test receptacle.

The latter described apparatus of the integral sterility testing system is suitable for membrane filtration sterility testing and is the preferred apparatus of the integral sterility testing system of the present invention.

The integral sterility testing system of the present invention is preferably susceptible to sterilisation, for example by gamma radiation. The system can thus be sterilised before use for screening for microorganism in a sample. A visual indicator that confirms verifiable sterilisation may be provided in the system of the present invention.

Moreover, after use, disposal of the sterility testing system is a viable alternative to sterilisation for re-use, because of the low cost of manufacture of the system. Accordingly, the sterility testing system of the present invention is preferably a one time use disposable system for screening for microorganism in a sample.

There is further provided by the present invention a method of screening for microorganisms in a sample, which method comprises:
a) providing a sample to be screened;
b) directing the sample to a test receptacle;
c) optionally directing wash medium to the test receptacle;
d) directing culture medium to the test receptacle;
e) incubating the test receptacle with culture medium therein under conditions that allow growth/proliferation of microorganisms present in the sample; and
f) monitoring for growth/proliferation of microorganisms in the culture medium, thereby providing an indication of the presence of microorganisms in the sample;
   wherein the wash and/or culture media (preferably wash and culture media) is directed to the test receptacle via a microorganism filter arrangement upstream of the test receptacle.

According to a second embodiment of the method of the present invention, there is provided a method of screening for microorganisms in a sample, which method comprises:
a) providing a sample to be screened;
b) directing the sample to a test receptacle having a sample microorganisms filter arrangement therein for filtering microorganisms from the sample;
c) allowing the sample to flow through the sample microorganism filter arrangement and out of the test receptacle;
d) optionally directing wash medium to the test receptacle to flush through the test receptacle and sample microorganisms filter arrangement therein;
e) directing culture medium to the test receptacle;
f) incubating the sample microorganism filter arrangement substantially in the culture medium under conditions that allow growth/proliferation of microorganisms present on the surface of the sample microorganism filter arrangement; and
g) monitoring for growth/proliferation of microorganisms in the culture medium, thereby providing an indication of the presence of microorganisms in the sample;
   wherein the wash and/or culture media (preferably wash and culture media) is directed to the test receptacle via a microorganism filter arrangement upstream of the test receptacle.

The method of the present invention is preferably performed using the apparatus of the present invention substantially as hereinbefore described.

The sample being screened for the presence of microorganisms such as bacteria, fungi and moulds, may be a pharmaceutical product or a food product, typically a parental pharmaceutical product, for example, a vaccine, therapeutic agent, intravenous infusion, eye drops or the like.

If the product is in solid form, such as a powder, discrete particles and the like, the product is preferably dissolved in a sterile liquid, for example a diluent or solvent, before being screened using the method or apparatus of the present invention.

Provided the dissolved product is capable of being filtered, the preferred method of screening is membrane filtration sterility testing according to the second embodiment of the method of the present invention.

In the second embodiment of the method of the present invention, microorganisms (or at least microorganisms above a certain size) present in the sample being screened are trapped and deposited on the surface of the sample microorganism filter arrangement in the test receptacle as the sample flows through the sample filter arrangement. The sample microorganism filter arrangement, is then incubated in culture medium under appropriate conditions for microbial growth/proliferation.

If the product to be tested cannot be filtered, for example an insoluble solid product, the product can be introduced into the test receptacle together with culture medium that has passed through the microorganism filter arrangement, typically so that the volume of the product is not more than 10% of the volume of the culture medium. The test receptacle with the inoculated culture medium therein is then incubated under appropriate conditions for microbial growth/proliferation.

The culture medium is typically a microorganism growth culture medium which provides optimal nutrients for growth/proliferation of particular microorganisms of interest. Examples of suitable culture media include soya-bean-casein digest medium for culture of aerobic bacteria and fungi and fluid thioglycollate medium for culture of anaerobic bacteria.

By growth/proliferation of microorganisms, we mean that the microorganisms are able to replicate and increase in number to an extent that enables detection of the microorganisms in the monitoring step of the method of the present invention.

The monitoring step typically comprises visual observation of a colour change or turbidity of the culture medium. A colour change or turbidity of the culture medium indicates growth/proliferation of microorganisms in the culture medium and thereby provides an indication of the presence of microorganisms in the sample.

Wash medium (typically a sterile solution, such as sterile distilled water, sterile peptone saline solution or the like) can optionally be used to flush through the test receptacle and preferably used to flush through the sample microorganism filter arrangement when present in the test receptacle.

The method of the present invention preferably includes running a negative control. The negative control typically involves substituting the sample to be screened with a sterile control fluid, such as sterile water or the like. The entire procedure (including incubation in substantially identical culture media under substantially the same incubation conditions) is processed in the same fashion as for the sample being screened. If at the conclusion of the control incubation period, there is indication of microorganism growth/proliferation, a review of the details of the procedure should be carried out to ascertain the source of contamination.

Additionally, or alternatively, one or more positive controls may be run. The positive control typically includes inoculating the culture medium with a small number of microorganisms, comprising as a minimum one aerobic bacteria and one anaerobic bacteria, and incubating the inoculated culture medium under conditions suitable for growth/ proliferation of the microorganisms. The culture medium is suitable for use in the method of the present invention if a clearly visible growth of the microorganisms occurs. A further positive control includes running the sterility test with the sample to be screened and separately with a sterile control fluid. A known concentration of microorganisms is added to the culture medium of both the sample test and the control. Comparison of the culture medium after incubation will indicate if the sample to be screened has any anti-microbial activity. If visible growth in the sample test is not comparable to the visible growth in the control, the product possesses antimicrobial activity and the conditions of the test need to be modified to substantially eliminate this antimicrobial activity.

The invention will now be described in specific embodiments, by way of example only, and with reference to the accompanying drawings in which:

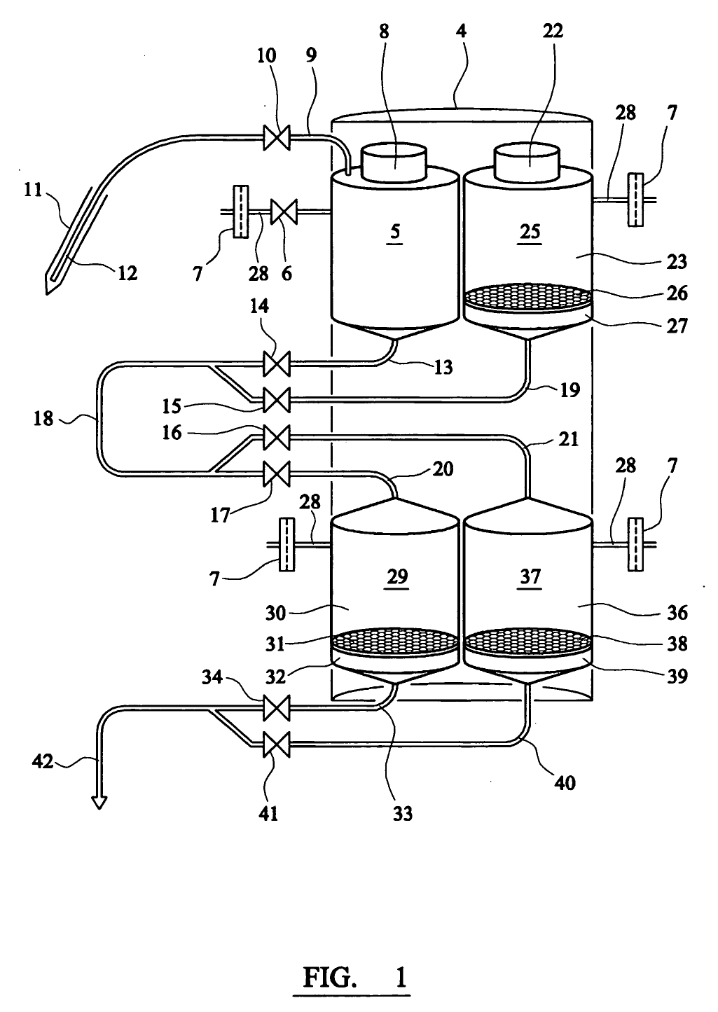

FIG. 1 shows a sterility testing system in accordance with the second aspect of the present invention;

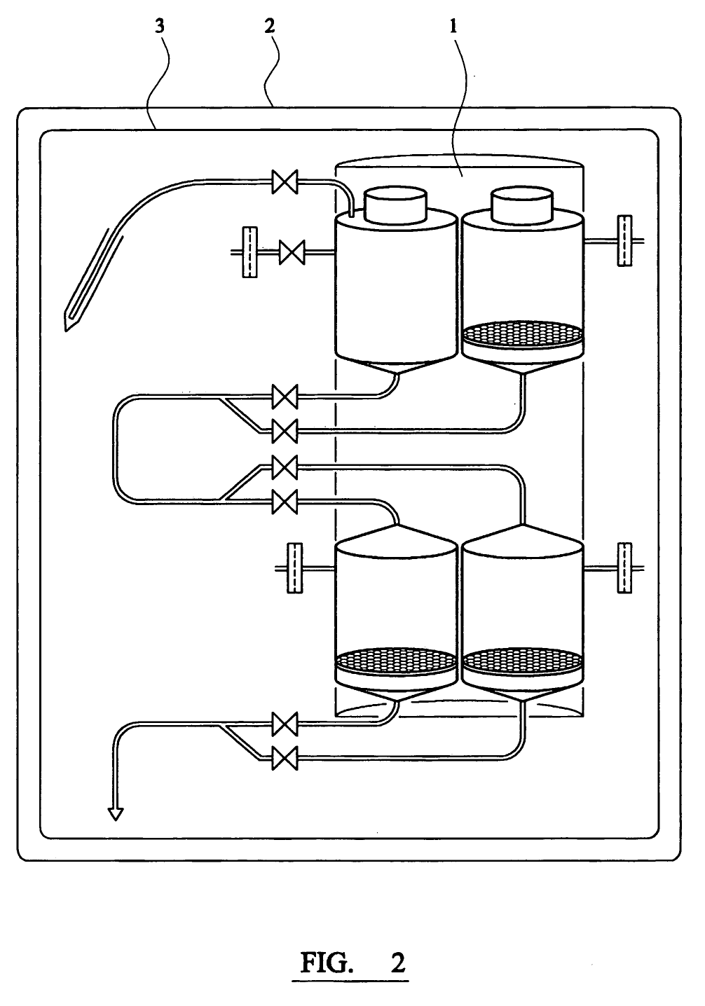

FIG. 2 shows the sterility testing system of FIG. 1 within sealed bags for transportation and storage;

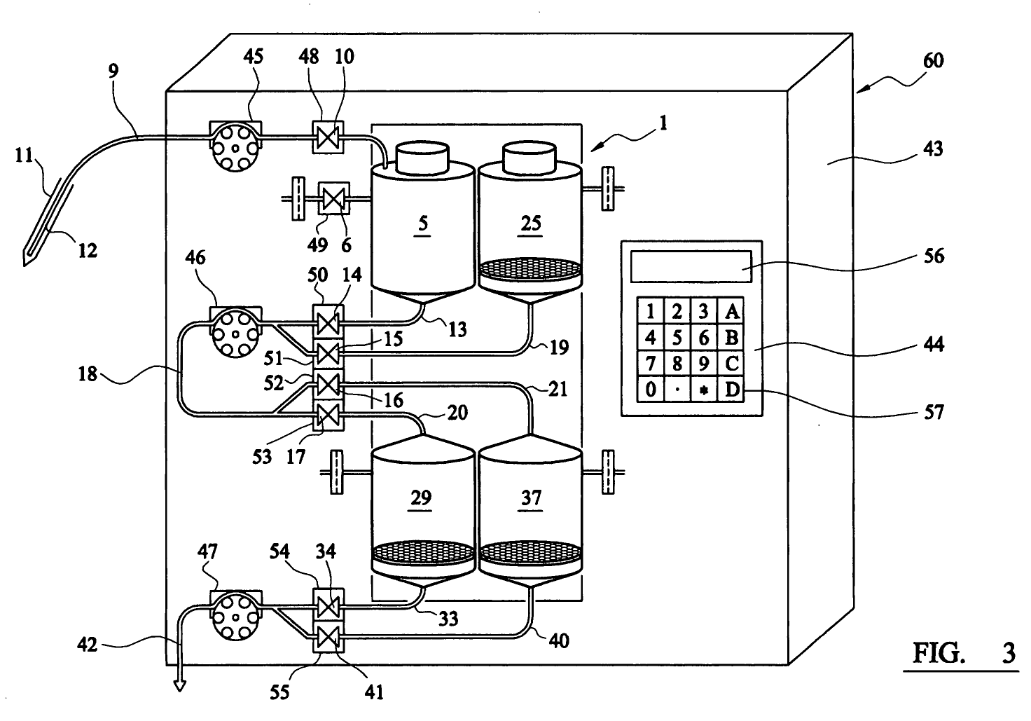

FIG. 3 shows the sterility testing system of FIG. 1 incorporated in an automated processor unit;

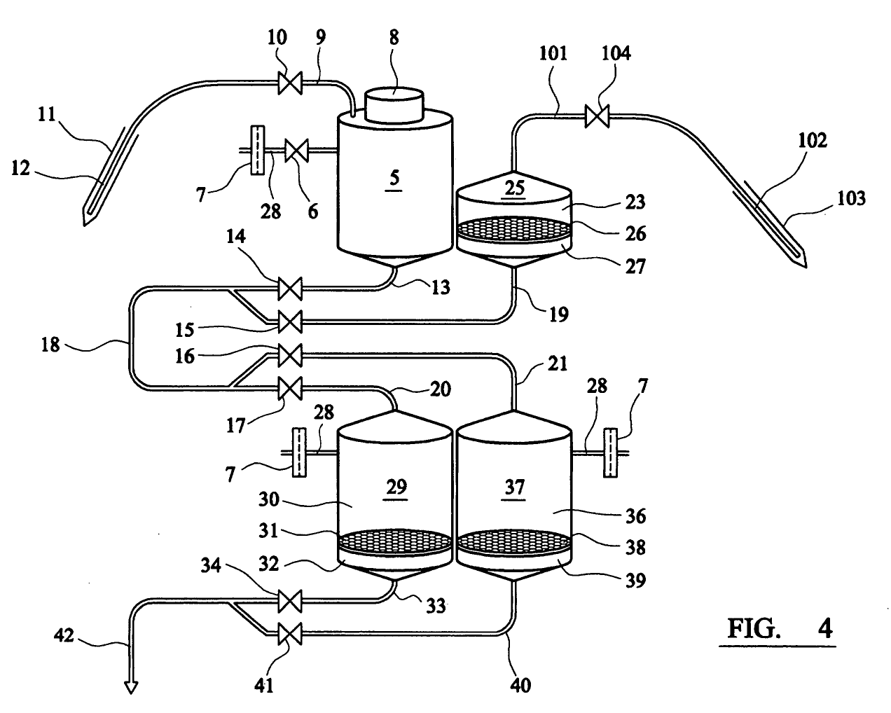

FIG. 4 shows an alternative embodiment of the sterility testing system in accordance with the second aspect of the present invention;

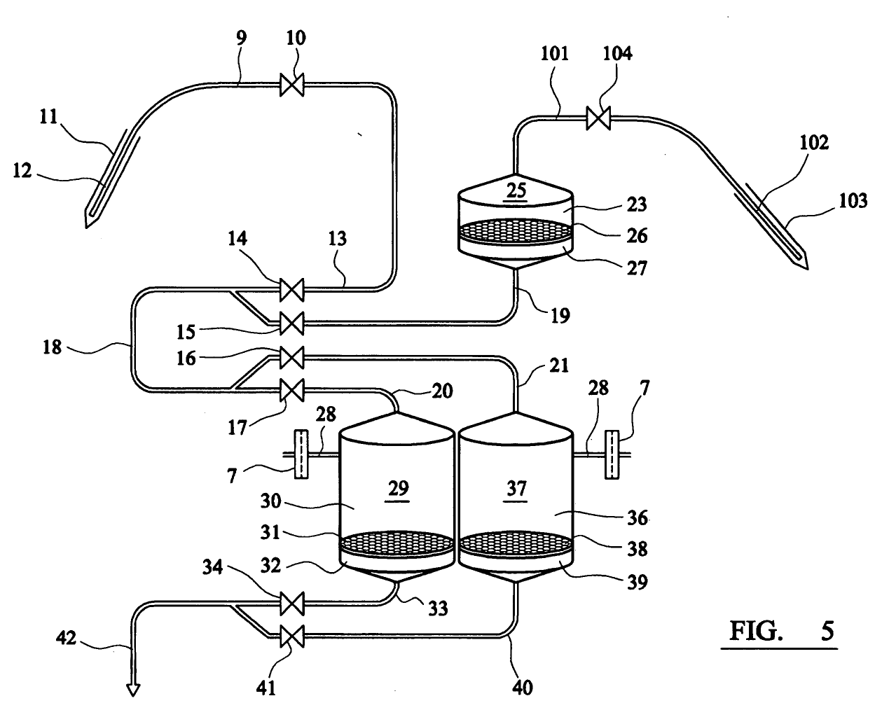

FIG. 5 shows a further alternative embodiment of the sterility testing system in accordance with the second aspect of the present invention;

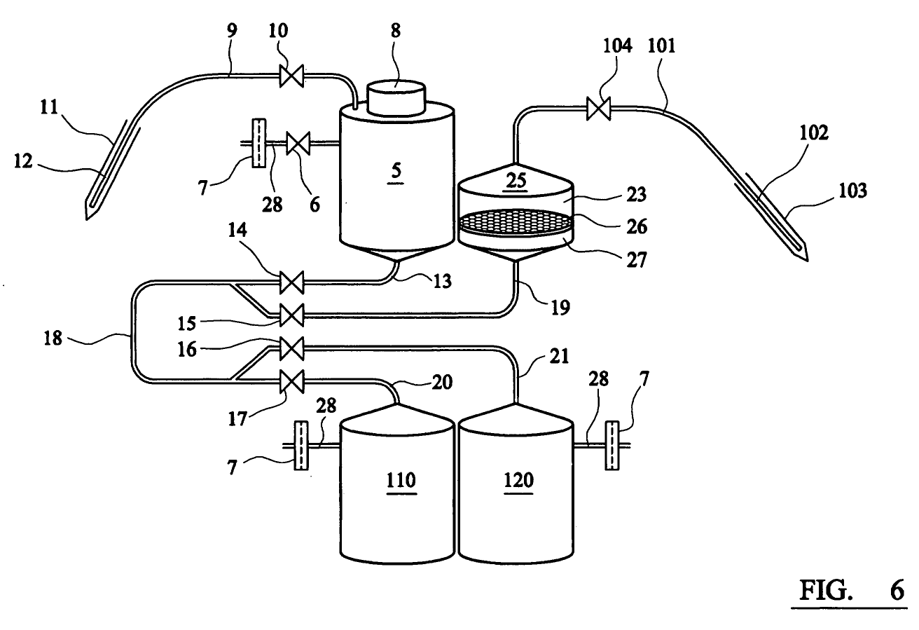

FIG. 6 shows a further sterility testing system in accordance with the present invention; and

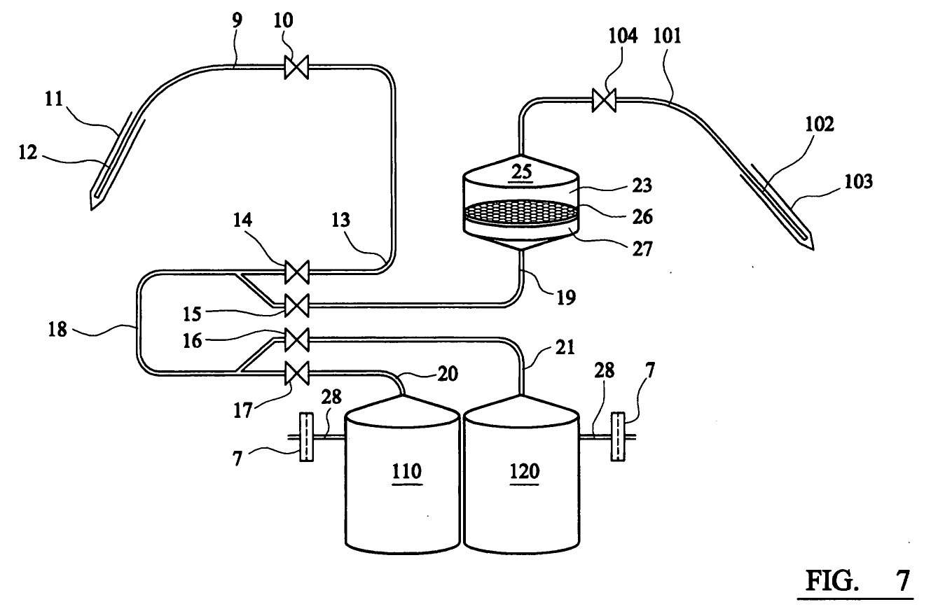

FIG. 7 shows a further sterility testing system in accordance with the present invention.

Referring to the drawings and initially to FIG. 1, there is shown an integral sterility testing system 1 comprising a support member 4 (typically of transparent plastics material) releasably supporting a sample receiving canister 5, a media receiving canister 25, a first test canister 29, and a second test canister 37.

Sample receiving canister 5 has a first and second inlet port for introduction of a sample into canister 5. The first inlet port is covered by a removable screw cap 8 and the second inlet port is connected to a flexible tube 9 that terminates in a cannula 12 protected by a removable plastic sheath 11. A pinch valve 10 acting externally on flexible tube 9 is provided to releasably seal flexible tube 9.

Media receiving canister 25 has an inlet port for introduction of wash media and growth culture media or the like into canister 25. The inlet port is covered by a removable screw cap 22. A membrane filter 26 (typically a filter no greater than 0.2 micron composed of cellulose acetate or cellulose nitrate) is sealed at its periphery to the inner wall of media receiving canister 25 generally parallel to the two ends of the canister 25. Membrane filter 26 is substantially the full diameter of media receiving canister 25 and divides the canister 25 into an upper chamber 23 and a lower chamber 27, the volume of the upper chamber 23 being greater than the volume of the lower chamber 27.

A further membrane filter 31 (typically a filter no greater than 0.45 micron composed of cellulose acetate or cellulose nitrate) is sealed at its periphery to the inner wall of first test canister 29 generally parallel to the two ends of the canister 29. Membrane filter 31 is substantially the full diameter of first test canister 29 and divides the canister 29 into an upper chamber 30 and a lower chamber 32, the volume of the upper chamber 30 being greater than the volume of the lower chamber 32.

A still further membrane filter 38 (typically a filter no greater than 0.45 micron composed of cellulose acetate or cellulose nitrate) is sealed at its periphery to the inner wall of second test canister 37 generally parallel to the two ends of the canister 37. Membrane filter 38 is substantially the full diameter of second test canister 37 and divides the canister 37 into an upper chamber 36 and a lower chamber 39, the volume of the upper chamber 36 being greater than the volume of the lower chamber 39.

The outlet port of sample receiving canister 5 and the outlet port of media receiving canister 25 are in fluid communication with the inlet port of first test canister 29 and the inlet port of second test canister 37 via a series of flexible tubes. The flexible tubes are provided with pinch valves that act externally on the flexible tubes to releasably seal the flexible tubes and thus control flow of fluid into test canisters 29, 37.

In more detail and following the direction of flow of fluids through sterility testing system 1, flexible tube 13 (releasably sealed with pinch valve 14) is connected to the outlet port of sample receiving canister 5 and flexible tube 19 (releasably sealed with pinch valve 15) is connected to the outlet port of media receiving canister 25. Flexible tubes 13 and 19 merge into flexible tube 18 which then splits into flexible tubes 20 and 21. Flexible tube 20 (releasably sealed with pinch valve 17) is connected to the inlet port of first test canister 29 and flexible tube 21 (releasably sealed with pinch valve 16) is connected to the inlet port of the second test canister 37.

In addition, the outlet port of first test canister 29 is connected to flexible tube 33 (releasably sealed with pinch valve 34) and the outlet port of second test canister 37 is connected to flexible tube 40 (releasably sealed with pinch valve 41). Flexible tubes 33 and 40 merge into flexible tube 42 which is connected to a fluid collection vessel (not shown).

Sample receiving canister 5, media receiving canister 25, first test canister 29 and second test canister 37 all have a vent port connected to a hydrophobic microporous vent filter 7 via a flexible tube 28. Hydrophobic microporous vent filter 7 filters all microorganisms above a specific size from the air flow through the filter 7. Flexible tube 28 may be provided with a pinch valve 6 (as shown in connection with the vent port of sample receiving canister 5). Pinch valve 6 acts externally on flexible tube 28 to releasably seal flexible tube 28 and thus control entry and exit of air and gases from the vent port.

Sample receiving canister 5, media receiving canister 25, first test canister 29 and second test canister 37 are typically of transparent plastic and may be provided with volumetric calibration marks (not shown) to indicate the volume of fluid in each canister. The different canisters may also be provided with different labels (not shown) for example a number, name or colour code, to aid identification of the different canisters and simplify the sterility testing procedure.

For transportation and storage, the fully assembled sterility testing system 1 is packaged within a sealed inner bag 3 within a sealed outer bag 2 as shown in FIG. 2. The packaged sterility testing system is subjected to sterilisation for example by gamma radiation. A visual indicator (not shown) that confirms verifiable sterilisation of the packaged sterility testing system is preferably provided to a component of the sterility testing system 1 or more preferably to the inside of inner bag 3 or the inside of outer bag 2. The outer bag 2 is typically opened on aseptic transfer to a clean room environment and the inner bag is opened at the sampling point.

The sterility testing system 1 is suitable for performing a membrane filtration sterility test in accordance with the second embodiment of the method of the present invention. To carry our this membrane filtration sterility test, the sterility testing system is prepared to receive a sample to be screened, by closing all pinch valves.

At the sampling point, the sample to be screened may be aseptically introduced into sample receiving canister 5 by a pipette or the like via the first inlet port simply by unscrewing screw cap 8 to expose the first inlet port. Screw cap 8 is replaced once the sample has been introduced.

Alternatively, or additionally, when the sample is a liquid, the sample may be aseptically pumped or aspirated into sample receiving canister 5 by removing plastic sheath 11, placing cannula 12 in a receptacle containing the sample (not shown) and opening pinch valve 10 to allow the sample to flow from canella 12, through flexible tube 9 and into canister 5 via the second inlet port. Once the sample has been introduced into canister 5, pinch valve 10 is closed to seal flexible tube 9.

If the sample to be screened comprises a product in solid form, such as a powder or discrete particles, the product may be dissolved in a sterile liquid, for example a diluent or solvent, before being introduced into the sample receiving canister via the first or second inlet port substantially as hereinbefore described. Alternatively, or additionally, a product in solid form may be introduced into the sample receiving canister 5 via the first inlet port substantially as hereinbefore described and dissolved in a sterile liquid, for example a diluent or solvent, by introducing the sterile liquid into the sample receiving canister 5 via the first or second inlet.

By closing pinch valves 10, 6 and 14 and screw cap 8, all entrances into and exits out of sample receiving canister 5 are sealed off. The sample can thus be stored within the sterility testing system 1 and transported if required, until such time and place that screening of the sample is carried out.

It is often required that a sample is screened for microorganisms that require different growth condition. In this case, a substantially equal amount of the sample is transferred from sample receiving canister 5 to the upper chamber 30 of first test canister 29 and the upper chamber 36 of second test canisters 37. Transfer of the sample is achieved by opening pinch valves 6 and 14 so that the sample can flow from the outlet port of sample receiving canister 5 through flexible tube 13 to flexible tube 18. Pinch valves 17 and 16 are used to control flow of the sample through flexible tubes 20 and 21 into upper chambers 30 and 36 respectively. Flow of the sample is induced by a peristaltic pump (not shown) or by application of a vacuum applied to upper chambers 30 and 36 by opening the hydrophobic microporous vent filters 7 of the first and second test canisters 29 and 37 respectively.

Once the sample has been transferred from sample receiving canister 5 in substantially equal amounts to first test canister 29 and second test canister 37, pinch valves 14, 16 and 17 are closed.

Pinch valve 34 is opened and the sample flows out of first test canister 29 into flexible tube 33 through flexible tube 42 to the fluid collection vessel (not shown). Pinch valve 41 is opened and the sample flows out of second test canister 37 into flexible tube 40 through flexible tube 42 to the fluid collection vessel (not shown). Flow of the sample is induced by a peristaltic pump (not shown) acting on flexible tube 42 or by application of a vacuum to the fluid collection vessel (not shown).

As the sample flows from the upper chamber 30 to the lower chamber 32 of first test canister 29 it passes through membrane filter 31. Similarly, as the sample flows from the upper chamber 36 to the lower chamber 39 of second test canister 37 it passes through membrane filter 38. Microorganisms (or at least microorganisms above a certain size) present in the sample are deposited on the surface of membrane filters 31 and 38.

Test canisters 29 and 37, and membrane filters 31 and 38 therein, are flushed through at least once with wash media (typically a sterile solution such as sterile distilled water or sterile peptone saline solution) in preparation for incubating the filters 31 and 38 in a suitable microorganism growth culture media. The wash media flushes away any sample remaining on the walls of test canisters 29 and 37 and on the surface of membrane filters 31 and 38 that could inhibit growth of microorganisms in the growth culture media.

Wash media is introduced into the upper chamber 23 of media receiving canister 25 via the inlet port by unscrewing screw cap 22. Screw cap 22 is replaced and pinch valve 15 is opened. Wash media flows from the upper chamber 23 to the lower chamber 27 of media receiving canister 25 passing through membrane filter 26, then out the outlet port of media receiving canister 25 into flexible tube 19 and through flexible tube 18. Pinch valves 17 and 16 are used to control flow of the wash media through flexible tubes 20 and 21 into upper chamber 30 of first test canister 29 and upper chamber 36 of second test canister 37, respectively. Flow of the wash media is induced as previously described in connection with induction of flow of the sample into test canisters 29 and 37.

Any foreign microorganisms present in the wash media are deposited onto the surface of membrane filter 26 and are thereby prevented from entering first and second test canister 29, 37. Advantageously, the wash media introduced into the test canisters 29 and 37 is therefore free from microorganisms that could otherwise contaminate the sterility testing procedure and lead to “false positive” test results that indicate the presence of microorganisms in the sample when none, in fact, are present.

Pinch valves 15, 16 and 17 are closed and pinch valves 34 and 41 are opened to allow the wash media to flow out of the first and second test canister 29, 37 and into the fluid collection vessel (not shown) as hereinbefore described in connection with drainage of the sample from test canisters 29 and 37.

The next step is to introduce a first type of microorganism growth culture media (media A) into the first test canister 29 and a second type of microorganism growth culture media (media B) into the second test canister 37 as follows.

All pinch valves of the sterility system 1 are closed. Media A is introduced into the upper chamber 23 of media receiving canister 25 via the inlet port by unscrewing screw cap 22. Screw cap 22 is replaced and pinch valve 15 is opened. Flow of media A is induced (as previously described) and media A flows from the upper chamber 23 to the lower chamber 27 of media receiving canister 25 through membrane filter 26. Media A then flows out the outlet port of media receiving canister 25 into flexible tube 19 and through flexible tube 18. Pinch valve 17 is opened to allow flow of media A through flexible tube 20 and into first test canister 29. Enough media A is introduced into first test canister 29 to submerge membrane filter 31.

All pinch valves of the sterility system 1 are again closed and media B is introduced into the upper chamber 23 of media receiving canister 25 via the inlet port by unscrewing screw cap 22. Screw cap 22 is replaced and pinch valve 15 is opened. Flow of media B is induced (as previously described) and media B flows from the upper chamber 23 to the lower chamber 27 of media receiving canister 25 through membrane filter 26. Media B then flows out the outlet port of media receiving canister 25 into flexible tube 19 and through flexible tube 18. Pinch valve 16 is opened to allow flow of media B through flexible tube 21 and into second test canister 37. Enough media B is introduced into second test canister 37 to submerge membrane filter 38.

As with the wash media, foreign microorganisms present in media A and media B are deposited onto the surface of membrane filter 26 and are thereby prevented from entering first test canister 29 and second test canister 37 respectively. Advantageously, media A and media B introduced into the first test canister 29 and second test canister 37 respectively is therefore free from microorganisms that could otherwise contaminate the sterility testing procedure and lead to “false positive” test results that indicate the presence of microorganisms in the sample when none, in fact, are present.

With pinch valves 16, 17 34 and 41 closed, first test canister 29 and second test canister 37 can be removed from support member 4 and incubated separately or together. Optimal incubation conditions are employed to allow growth/proliferation of microorganism in media A and media B. Visual observation of a colour change or turbidity of media A and/or media B indicates the presence of microorganisms in the sample.

As in any sterility test system, a negative control may be run in which a sterile control fluid is substituted for the sample and the entire procedure (including incubation in identical microorganism growth culture media) is processed in the same fashion as for the actual sample to be screened. If at the conclusion of the incubation period, microorganism growth proliferation is observed in either of the canisters, a review of the details of the procedure must be carried out to ascertain the source of contamination.

The components of sterility testing system 1 are inexpensive and easy to manufacture enabling the system 1 to be a one time use disposable system.

The sterility testing system 1 can be incorporated in an automated process unit 60 as shown in FIG. 3. The automated process unit 60 comprises a housing 43 incorporating a control unit 44, a number of peristaltic pump heads 45, 46, 47 and a number of pinch valve actuators 48, 49, 50, 51, 52, 53, 54, 55.

Sterility testing system 1 is releasably supported within housing 43 such that, peristaltic pump head 45 interacts with flexible tube 9 to induce flow of sample from the cannula 12 to sample receiving canister 5; peristaltic pump head 46 interacts with flexible tube 18 to induce flow of fluids from sample receiving canister 5 and media receiving canister 25 to first test canister 29 and second test canister 37; and peristaltic pump head 47 interacts with flexible tube 42 to induce flow of fluids from first test canister 29 and second test canister 37 to a fluid collection vessel (not shown). Furthermore, pinch valve actuators 48, 49, 50, 51, 52, 53, 54, 55 interact with pinch valves 10, 6, 14, 15, 16, 17, 34, 41 respectively.

Control unit 44 is programmable and linked to the peristaltic pump heads 45, 46, 47 and the pinch valve actuators 48, 49, 50, 51, 52, 53, 54, 55 enabling automated synchronous operation of the peristaltic pumps and pinch valves to control flow of sample or media through the sterility testing system 1 and release of air and gases from sample receiving canister 5. Control unit 44 comprises a keyboard 57 and a visual display unit 56 to provide an interface for a human operator.

The sterility testing system 1 of FIGS. 4 to 7 is similar to the sterility testing system 1 of FIG. 1 and like reference numerals are used to denote like parts.

In FIGS. 4 to 7, the inlet port of media receiving cannister 25 is connected to a flexible tube 101 which terminates in a cannula 102 protected by a removable plastic sheath 103. A pinch valve 104 acting externally on flexible tube 101 is provided to releasably seal flexible tube 101.

In use, wash and/or culture medium is introduced into media receiving cannister 25 by removing plastic sheath 103 and pushing cannula 102 through the septum of a bottle of wash or culture medium (not shown). Pinch valve 104 is opened and wash/culture media is aseptically pumped or aspirated into media receiving cannister 25. Once the wash/culture media has been introduced into cannister 25, pinch valve 104 is closed to seal flexible tube 101.

The sterility testing system 1 of FIGS. 5 and 7 has no sample receiving cannister 5, the sample to be screened is introduced directly into test canisters 29 and 37 via cannula 12 connected to flexible tube 10, flexible tube 10 being connected directly to flexible tube 13.

The sample is aseptically pumped or aspirated from a receptacle containing the sample to test receptacles using cannula 12.

The sterility testing system 1 of FIGS. 6 and 7 are suitable for direct inoculation sterility testing when the sample to be tested is insoluble and therefore cannot be filtered. In use, the sample to be tested is introduced into first test cannister 110 and second test cannister 120 substantially as hereinbefore described in connection with FIGS. 1 and 5. Test canisters 110 and 120 have an inlet connected to flexible tubes 20 and 21 respectively but no outlet, therefore the sample to be screened is trapped in test canisters 110 and 120. Before, during or after introduction of sample into test canisters 110 and 120, culture medium A is introduced into first test canister 110 and culture media B is introduced into second test cannister 120 via media receiving cannister 25 substantially as hereinbefore described.

The volume of sample is typically not more than 10% of the volume of culture media A and B in the test canisters 110 and 120 respectively. When it is necessary to use a large volume of sample to be tested, it may be preferable to use a concentrated culture medium prepared in such a way that it takes account of the subsequent dilution.

Culture media A and B introduced into test canisters 110 and 120 respectively has passed through membrane filter 26 and is therefore advantageously free from microorganisms that could otherwise contaminate the direct inoculation sterility testing procedure and lead to ‘false positive’ test results that indicate the presence of microorganisms in the sample when none, in fact, are present.

The inoculated culture media A and B within test canisters 110 and 120 respectively is incubated under conditions suitable for microbial growth/proliferation. Visual observation of a colour change or turbidity of media A and/or media B indicates the presence of microorganisms in the sample.

The sterility testing system 1 of FIGS. 4 to 7 can be transported and stored within the sealed bags 2 and 3 of FIG. 2. Furthermore, the sterility testing system 1 of FIGS. 4 to 7 can be incorporated in the automated processor unit 60 of FIG. 3.

Any number of sample receiving canisters, media receiving canisters, or test canisters can be incorporated in the sterility testing system in accordance with the present invention.

1. An apparatus for screening for microorganisms in a sample, which apparatus comprises:
i) a test receptacle for growth/proliferation of microorganisms present in the sample;
ii) a sample flowpath to direct the sample to the test receptacle; and
iii) a further flowpath to direct wash and/or culture media to the test receptacle;
wherein, the further flowpath has a microorganism filter arrangement upstream of the test receptacle.

2. Apparatus according to claim 1, wherein the further flowpath directs wash and culture media to the test receptacle.

3. Apparatus according to claim 1, including a length of confluence flowpath comprising both the downstream portion of the sample flowpath and the downstream portion of the further flowpath, the confluence flowpath being connected to the test receptacle, wherein the microorganism filter arrangement is provided in the further flowpath branch upstream of the confluence flowpath and not in line with the sample flowpath.

4. Apparatus according to claim 1, wherein the sample flowpath includes a sample receptacle arranged and configured to receive the sample to be screened, the sample receptacle being in fluid communication with and upstream of the test receptacle.

5. Apparatus according to claim 1, wherein the further flowpath includes a media receptacle arranged and configured to receiving wash and/or culture media, the media receptacle being in fluid communication with and upstream of the test receptacle, in which the microorganism filter arrangement is provided: c) internally of the media receptacle; and/or d) in the further flowpath downstream of the media receptacle but not in-line with the sample flowpath.

6. Apparatus according to claim 5, wherein the microorganism filter arrangement is provided internally of the media receptacle.

7. Apparatus according to claim 5, wherein the media receptacle is arranged and configured to receive both wash and culture media.

8. Apparatus according to claim 1, including two or more test receptacles.

9. Apparatus according to claim 1, wherein the test receptacle has a sample microorganism filter arrangement therein, for filtering microorganisms from the sample.

10. Apparatus for screening for microorganisms in a sample, which apparatus comprises:
i) a test receptacle having a sample microorganism filter arrangement therein for filtering microorganisms from the sample;
ii) a sample flowpath to direct the sample to the test receptacle; and
iii) a further flowpath to direct wash and/or culture media to the test receptacle; wherein the further flowpath has a microorganism filter arrangement upstream of the test receptacle.

11. Apparatus according to claim 10 including a support member that releasably supports the test receptacle.

12. Apparatus according to claim 10 including a control system used to automate flow of fluids to the test receptacle.

13. Apparatus according to claim 10 packaged within at least one substantially sealed bag.

14. Apparatus according to claim 13 packaged within a substantially sealed inner bag, the inner bag being within a substantially sealed outer bag.

15. An integral sterility testing system comprising an apparatus for screening for microorganisms in a sample, the apparatus being substantially sealed within at least one bag.

16. The system according to claim 15, wherein the apparatus is substantially sealed within an inner bag, the inner bag being substantially sealed within an outer bag.

17. The system according to claim 15, wherein the apparatus comprises:
i) a test receptacle for growth/proliferation of microorganisms present in the sample being screened;
ii) a sample flowpath to direct the sample to the test receptacle; and iii) a further flowpath to direct wash and/or culture media to the test receptacle; the further flowpath having a microorganism filter arrangement upstream of the test receptacle.

18. The system according to claim 15, wherein the apparatus comprises:
i) a test receptacle having a sample microorganism filter arrangement therein for filtering microorganisms from the sample being screened;
ii) a sample flowpath to direct the sample to the test receptacle; and
iii) a further flowpath to direct wash and/or culture media to the test receptacle; the further flowpath having a microorganism filter arrangement upstream of the test receptacle.

19. The system according to claim 15, wherein the system is susceptible to sterilisation.

20. The system according to claim 15, wherein the system is a one time use disposable system for screening for microorganism in a sample.

21. A method of screening for microorganisms in a sample, which method comprises:
a) providing a sample to be screened;
b) directing the sample to a test receptacle;
c) optionally directing wash medium to the test receptacle;
d) directing culture medium to the test receptacle;
e) incubating the test receptacle with culture medium therein under conditions that allow growth/proliferation of microorganisms present in the sample; and
f) monitoring for growth/proliferation of microorganisms in the culture medium, thereby providing an indication of the presence of microorganisms in the sample; wherein the wash and/or culture media is directed to the test receptacle via a microorganism filter arrangement upstream of the test receptacle.

22. A method of screening for microorganisms in a sample, which method comprises:
a) providing a sample to be screened;
b) directing the sample to a test receptacle having a sample microorganisms filter arrangement therein for filtering microorganisms from the sample;
c) allowing the sample to flow through the sample microorganism filter arrangement and out of the test receptacle;
d) optionally directing wash medium to the test receptacle to flush through the test receptacle and sample microorganisms filter arrangement therein;
e) directing culture medium to the test receptacle;
f) incubating the sample microorganism filter arrangement substantially in the culture medium under conditions that allow growth/proliferation of microorganisms present on the surface of the sample microorganism filter arrangement; and
g) monitoring for growth/proliferation of microorganisms in the culture medium, thereby providing an indication of the presence of microorganisms in the sample; wherein the wash and/or culture media is directed to the test receptacle via a microorganism filter arrangement upstream of the test receptacle.

23. The method according to claim 21 wherein the test receptacle is provided in an apparatus comprising:
i) a test receptacle for growth/proliferation of microorganisms present in the sample;
ii) a sample flowpath to direct the sample to the test receptacle; and
iii) a further flowpath to direct wash and/or culture media to the test receptacle;
wherein, the further flowpath has a microorganism filter arrangement upstream of the test receptacle.

24. The method according claim 28, wherein wash and culture media are directed to the test receptacle via the microorganism filter arrangement.

25. The method according to claim 21 including running a control.

26-27. (canceled)

28. The method according to claim 22 wherein the test receptacle is provided in an apparatus comprising:
i) a test receptacle having a sample microorganism filter arrangement therein for filtering microorganisms from the sample;
ii) a sample flowpath to direct the sample to the test receptacle; and
iii) a further flowpath to direct wash and/or culture media to the test receptacle;
wherein, the further flowpath has a microorganism filter arrangement upstream of the test receptacle.

29. Apparatus according to claim 2, including a length of confluence flowpath comprising both the downstream portion of the sample flowpath and the downstream portion of the further flowpath, the confluence flowpath being connected to the test receptacle, wherein the microorganism filter arrangement is provided in the further flowpath branch upstream of the confluence flowpath and not in line with the sample flowpath.

30. Apparatus according to claim 1 including a support member that releasably supports the test receptacle.

31. The system according to claim 16, wherein the apparatus comprises:
i) a test receptacle for growth/proliferation of microorganisms present in the sample being screened;
ii) a sample flowpath to direct the sample to the test receptacle; and iii) a further flowpath to direct wash and/or culture media to the test receptacle; the further flowpath having a microorganism filter arrangement upstream of the test receptacle.

32. The system according to claim 16, wherein the apparatus comprises:
i) a test receptacle having a sample microorganism filter arrangement therein for filtering microorganisms from the sample being screened;
ii) a sample flowpath to direct the sample to the test receptacle; and
iii) a further flowpath to direct wash and/or culture media to the test receptacle; the further flowpath having a microorganism filter arrangement upstream of the test receptacle.
